(19) United States
(12) Patent Application Publication
(10) Pub. No.: US 2005/0227920 A1
(43) Pub. Date: Oct. 13, 2005

(54) Methods for production of recombinant vascular endothelial cell growth inhibitor

(76) Inventor: Xinli Lin, Piedmont, CA (US)

Correspondence Address: MORRISON & FOERSTER LLP, 755 PAGE MILL RD, PALO ALTO, CA, 94304-1018, US

(21) Appl. No.: 11/011,406

(22) Filed: Dec. 13, 2004

Related U.S. Application Data
(60) Provisional application No. 60/528,983, filed on Dec. 11, 2003.

(51) Int. Cl. A61K 38/18; C07K 14/475

(52) U.S. Cl. 514/12; 530/399

(57) ABSTRACT

Methods of producing properly folded recombinant VEGI polypeptide are provided. Denatured recombinant VEGI polypeptide is refolded by first solubilizing the polypeptide with a chaotroph at high pH, followed by refolding in the presence of reduced concentrations of chaotroph and in the presence of a detergent while the pH is slowly reduced.

### CROSS-REFERENCES TO RELATED APPLICATIONS

This application claims the priority benefit of provisional patent application U.S. Ser. No. 60/528,983, filed Dec. 11, 2003, which is incorporated herein by reference in its entirety.

### STATEMENT REGARDING FEDERALLY SPONSORED RESEARCH OR DEVELOPMENT

This invention was made with U.S. Government support under National Institutes of Health grant NIH-CA102181. The U.S. Government may have certain rights in this invention.

### FIELD OF THE INVENTION

This invention relates to methods for producing recombinant vascular endothelial cell growth inhibitor (VEGI) polypeptides.

### BACKGROUND OF THE INVENTION

Vascular endothelial cell growth inhibitor (VEGI) is an endothelial cell-specific gene product. Four isoforms of human VEGI have been reported: The first form of VEGI discovered is 174 amino acids in length; two different forms of 192 amino acid residues and one of 251 amino acid residues are later discovered. See Zhai et al., Int. J. Cancer 82:131-136 (1999); Zhai et al., FASEB J. 13: 181-189 (1999); Chew et al., FASEB J. 16: 742-744 (2002); PCT WO03/039491; U.S. patent application Pub. No. 2003/0170242. All isoforms are splicing variants arising from a common gene. The four isoforms differ in their N-terminal regions but share an identical core of 151 amino acids encoding the rest of the protein.

A comparison of the sequences of the four isoforms indicates that they share 20-30% identity with the tumor necrosis factor (TNF) superfamily of proteins. The roles of the different isoforms have not been clearly delineated and is undoubtedly subtle and complex—with both membrane bound and secreted forms of the molecule being reported. All of the evidence reported thus far seems to point to secreted forms of VEGI-174 inhibits tumor cell growth and initiates apoptosis. Hydrophobicity profiling of VEGI-174 implies that it is a typical type II transmembrane protein, with amino acids 29-174 constituting the extracellular domain. Full length VEGI-174 does not have any effect on tumor growth when overexpressed in cancer cells, nor does it inhibit endothelial cells when transfected into these cells. See Zhai et al., FASEB J. 13: 181-189 (1999); Chew et al., FASEB J. 16: 742-744 (2002). Several members of the TNF family, including TNF and Fas ligand have been shown to be cleaved from the membrane and function as soluble proteins. See Bjornberg et al., Scand J. Immunol. 42: 418-424 (1995); Kayagaki et al., J. Exp. Med. 182: 1777-83 (1995). This has not yet been demonstrated for VEGI-174. Nevertheless, an artificial recombinant secretory form of this VEGI-174 (s-VEGI) comprising only the extracellular domain of VEGI-174 and a secretion signal peptide derived from a secretory protein inhibited tumor growth when overexpressed in cancer cells. See Zhai et al., Int. J. Cancer 82:131-136 (1999); U.S. patent application Pub. No. 2002/0111325. VEGI-251, the most abundant isoform, possesses a putative secretory signal peptide. Over-expression of VEGI-251 causes endothelial cell apoptosis and growth inhibition. PCT WO03/039491; U.S. patent application Pub. No. 2003/0170242. Similarly, recombinant VEGI that contains the 151 amino acid core sequence shared by all forms of VEGI has been shown to initiate apoptosis and block tumor cell growth, albeit with low potency. See Wang, et al., Acta Biochimica et Biophysica Sinica 32(5): 485-489 (2000). It has been reported that recombinantly produced and refolded VEGI-192A inhibited proliferation of adult bovine aortic endothelial (ABAE) cells in vitro. PCT WO03/039491; U.S. patent application Pub. No. 2003/0170242. Methods for refolding proteins have been reported in U.S. Pat. No. 6,583,268; PCT WO 2004/094344.

All patents, patent applications, and publications cited herein are hereby incorporated by reference in their entirety. It should be noted that reference to a publication in this Background section is not an admission that the publication constitutes prior art to the instant invention.

### BRIEF SUMMARY OF THE INVENTION

The invention provides a new refolding method to produce VEGI in active form. The instant methods utilize, in some embodiments, crude bacterially-produced VEGI polypeptide (e.g., either from cell paste or inclusion bodies), and generate correctly folded, highly active VEGI polypeptides using only a small number of steps. As described herein, correctly folded, highly active VEGI-192A was generated using crude bacterially-produced VEGI-192A (with or without a N-terminal His-Tag) (e.g., either cell paste or inclusion bodies). The recombinant protein produced according to the methods described herein exhibited IC₅₀ of 0.24-2.4 nM (6-60 ng/ml) for VEGI-192A with a His-Tag and 2.4-24 nM for VEGI-192A without a His-Tag in inhibiting endothelial cell proliferation in vitro. Biologically active VEGI-174 and amino-terminal truncated VEGI-251 (amino acids 86-251 fragment) were also generated using the same method.

Generally, the invention provides methods of producing a biologically active VEGI (such as VEGI-192A) from VEGI containing inclusion bodies from bacteria cells (such as E. coli) by solubilizing the inclusion body protein in a buffer containing disulfide reducing agents and a high concentration of chaotroph (e.g., 8 M urea or 6 M guanidine HCl) at high pH (i.e., greater than about pH 9), and refolding by reducing the chaotroph concentration and slowly reducing the pH to near-neutral (i.e., pH 7.5-8.5) in the presence of a detergent (e.g., sodium lauroyl sarcosine, trimethylamine N-oxidedihydrate (TMAO), cetyltrimethylammunium bromide (CTAB), or any combination of them). The refolded VEGI (such as VEGI-192A) protein may then be purified.

The invention provides methods for producing refolded recombinant VEGI (such as VEGI-192A) by solubilizing denatured VEGI protein with a solubilization buffer containing a high concentration of chaotroph, a reducing agent, and having a pH of about 9.0 to about 11.0, to produce a solubilized VEGI solution, rapidly diluting the solubilized VEGI solution with refolding buffer by adding the solubilized VEGI solution into the refolding buffer containing a detergent to produce a diluted solubilized VEGI solution, and reducing the pH of the diluted solubilized VEGI solution to a pH of about 7.5 to about 8.5, wherein said pH reducing is carried out over a period of at least about 20 hours, thereby producing refolded VEGI.

In certain embodiments, the VEGI is a human VEGI. In some embodiments, the VEGI comprises amino acid sequence of SEQ ID NO: 1. In some embodiments, the VEGI comprises amino acid sequence of SEQ ID NO:6. In some embodiments, the VEGI comprises amino acids from 24-174 of SEQ ID NO:6. In some embodiments, the VEGI comprises amino acids from 86-251 of SEQ ID NO:4.

In certain embodiments, the chaotroph is urea, which may be at about 8 M concentration. In other embodiments, the chaotroph is guanidine hydrochloride, which may be at about 6 M concentration.

In certain embodiments, the solubilizing buffer is about pH 10. In certain embodiments, the solubilizing buffer is about pH 10.5. In certain embodiments, the solubilizing buffer is about pH 10.8. In certain embodiments, the solubilizing buffer is about pH 10.0 to about pH 10.5. In certain embodiments, the solubilizing buffer is about pH 10.0 to about pH 10.8.

In certain embodiments, the pH of the diluted solubilized VEGI solution is reduced to about pH 8.0.

In certain embodiments, the method further comprises adjusting the A₂₈₀ of the solubilized VEGI solution to about 2.0 to about 5.0 before rapidly diluting the solubilized VEGI solution. In certain embodiments, the A₂₈₀ of the solubilized VEGI solution is adjusted by diluting with the solubilization buffer, for example, a solubilization buffer comprising about 8 M urea, about 0.1 M Tris, about 1 mM glycine, about 10 mM β-mercaptoethanol, about 10 mM dithiothreitol (DTT), about 1 mM reduced glutathion (GSH), and about 0.1 mM oxidized glutathion (GSSG) at pH about 10.0 to about 10.8.

In certain embodiments, the solubilized VEGI solution is diluted about twenty-fold into the refolding buffer.

In certain embodiments, the refolding buffer comprises one or more detergent. In certain embodiments, the detergent is sodium lauroyl sarcosine, trimethylamine N-oxidedihydrate (TMAO), cetyltrimethylammunium bromide (CTAB), or any combination of these detergents.

The invention may comprise additional steps at the beginning of the process. Thus, in certain embodiments the method includes the preliminary step of lysing bacterial host cells comprising denatured VEGI protein and collecting said denatured VEGI protein. Certain additional embodiments also include washing the denatured VEGI protein.

The invention may also comprise additional steps at the end of the process. Thus, certain embodiments also include purification of the refolded VEGI (such as VEGI-192A), such as by size exclusion chromatography (SEC), affinity chromatography (such as Ni⁺-affinity chromatography) or a combination of these steps, such as both SEC followed and affinity chromatography, which can be used in either order. In certain embodiments, buffer exchange was performed to remove the detergent in the refolding buffer before purification.

The VEGI (such as VEGI-192A) thus produced may have an IC₅₀ of at least about any of 1000 ng/ml, 100 ng/ml, 60 ng/ml, 40 ng/ml, 20 ng/ml, 12 ng/ml, or 6 ng/ml in inhibiting vascular endothelial cell proliferation in vitro.

In an exemplary embodiment, denatured VEGI (such as VEGI-192A) polypeptide is solubilized with a buffer comprising about 8 M urea and about 100 mM beta-mercaptoethanol at about pH 10.5, to produce solubilized VEGI polypeptide, concentration adjusted to about 1.7 mg/mL, rapidly diluted about twenty-fold into refolding buffer comprising about 20 mM Tris, sodium lauroyl sarcosine, trimethylamine N-oxidedihydrate (TMAO), and cetyltrimethylammunium bromide (CTAB), pH 10.5; and the pH of the diluted solubilized VEGI polypeptide is adjusted to about pH 8 over a period of at least about 20 hours to 4 days. In some embodiments, the refolding buffer comprises 1.36 mM Sodium Lauroyl Sarcosine, 0.009 mM Trimethylamine N-oxidedihydrate, 0.005 mM Cetyltrimethylammonium Bromide.

The invention also provides properly folded VEGI (such as VEGI-192A) and VEGI produced by the instant methods.

### BRIEF DESCRIPTION OF THE SEVERAL VIEWS OF THE DRAWING(S)

FIG. 1 shows that purification of refolded VEGI-192A (with His-Tag) and endothelial-cell (ABAE) growth arrest assay. FIG. 1A shows Sephacryl S-300 column chromatography of refolded VEGI-192A. The column was equilibrated and run with 20 mM Tris, 0.2 M NaCl, 0.4 M urea, pH 8.0. On the top of the peaks, 1, 2, and 3 represent three pools of fractions. FIG. 1B shows nonreducing SDS PAGE analysis of chromatography fractions, pools 1, 2, and 3, shown in FIG. 1A. FIG. 1C shows that endothelial cell growth arrest assay of pool 3, indicating the 50% inhibition concentration (IC50) is 12 ng/ml (0.49 nM).

FIG. 2 shows affinity purification of VEGI-192A (with His-Tag). FIG. 2A shows Ni⁺-affinity column purification of refolded VEGI-192A. Arrow indicates VEGI-192A peak. FIG. 2B shows SDS-PAGE of pooled and dialyzed sample from FIG. 2A. 1, non-reduced; 2, reduced.

FIG. 3 shows that purification of refolded VEGI-192A with His-Tag and with no tag and endothelial-cell (ABAE) growth arrest assay. FIG. 3A shows nonreducing SDS-PAGE of refolded VEGI-192A. VEGI-192A with no tag was loaded on lane 1; and VEGI-192A with His-Tag was loaded on lane 2. FIG. 3B shows endothelial cell growth arrest assay of VEGI-192A with no tag. FIG. 3C shows endothelial cell growth arrest assay of VEGI-192A with His-Tag.

### DETAILED DESCRIPTION OF THE INVENTION

The instant invention provides methods for the production of recombinant, biologically active VEGI (such as VEGI-192A).

The practice of the present invention will employ, unless otherwise indicated, conventional techniques of immunology, molecular biology, microbiology, cell biology and recombinant DNA, which are within the skill of the art. See, e.g., Molecular Cloning: a laboratory manual, 2^{nd} edition Sambrook, et al. (1989); Current Protocols In Molecular Biology F. M. Ausubel, et al. eds., (1987); the series Methods In Enzymology, Academic Press, Inc.; PCR 2: A Practical Approach, M. J. MacPherson, B. D. Hames and G. R. Taylor, eds. (1995), and Antibodies, A Laboratory Manual, Harlow and Lane, eds. (1988).

It should be noted that, as used herein, the singular form “a”, “an”, and “the” includes plural references unless indicated otherwise. Additionally, as used herein, the term “comprising” and its cognates are used in their inclusive sense; that is, equivalent to the term “including” and its corresponding cognates, in accordance with well-established principles of patent law.

VEGI (used interchangeably with VEGI protein and VEGI polypeptide) includes any naturally occurring species (such as full length from any mammalian, e.g., human, farm animals, sport animals, pets, primates, horses, dogs, cats, mice and rats), biologically active polypeptide fragments (such as fragment described in WO 03/039491 and U.S. patent Pub. No. 2003/017242), and variants (including naturally occurring and non-naturally occurring), including functionally equivalent variants which do not significantly affect their biological properties and variants which have enhanced or decreased activity (e.g., inhibiting endothelian cell growth). Examples of variants include VEGI with one or more amino acid substitution (e.g., conservative substitution), one or more deletions or additions of amino acids which do not significantly change the folding or functional activity of the protein.

Human VEGI protein includes VEGI-174, VEGI-251, VEGI-192A and VEGI-192B. Amino acid sequences of different isoforms of human VEGI are shown in Table 2 and described in U.S. Pub. No. 2003/0170242, PCT WO 03/039491, and WO 99/23105. In some embodiments, VEGI protein comprises amino acid sequence of SEQ ID NO: 1. In some embodiments, the VEGI comprises amino acid sequence of SEQ ID NO:6. In some embodiments, the VEGI comprises amino acid sequences of various amino-terminal truncated VEGI fragment, e.g., 57-251, 68-251, 86-251, or 100-251 of VEGI-251. In some embodiments, the VEGI comprises amino acids from 24-174 of SEQ ID NO:6. In some embodiments, the VEGI comprises amino acids from 86-251 of SEQ ID NO:4. VEGI embodiments include fusion proteins (N-terminal fusion or C-terminal fusion), for example, N-terminal fusion protein shown in Table 3. Nucleotide sequence and amino acid sequences of human VEGI-192A are also described in PCT WO03/039491 and U.S. patent application Pub. No. 2003/0170242.

TABLE 1

Amino acid seciuence of human VEGI-192A
(SEQ ID NO:1)

MQLTKGRLHFSHPLSHTKHISPFVTDAPLRADGDKPRAHLTVVRQTPTQ
HFKNQFPALHWEHELGLAFTKNRMNYTNKFLLIPESGDYFIYSQVTFRG
MTSECSEIRQAGRPNKPDSITVVITKVTDSYPEPTQLLMGTKSVCEVGS
NWFQPIYLGAMFSLQEGDKLMVNVSDISLVDYTKEDKTFFGAFLL

TABLE 2

Alignment of the amino acid sequences of the four VEGI isoforms *:
(SEQ ID NOS:4, 1, 5, 6)

| | | |
|---|---|---|
| VEGI - 251 | MAEDLGLSFGETASVEMLPEHGSCRPKARSSSARWALTCCLVLLPFLAGLTTYLLVSQL | 59 |
| VEGI - 251 | RAQGEACVQFQALKGQEFAPSNQQVYAPLRADGDKPRAHLTVVRQTPTQHFKNQFPALHW | 119 |
| VEGI - 192A | MQLTKGRLHFSHPLSHTKHISPFVTDAPLRADGDKPRAHLTVVRQTPTQHFKNQFPALHW | 60 |
| VEGI - 192B | METSQEHQGPSDIHRIPWSWGQRNSHAPLRADGDKPRAHLTVVRQTPTQHFKNQFPALHW | 60 |
| VEGI - 174 | MRRFLSKVYSFPMRKLILFLVFPVVRQTPTQHEFKNQFPALHW | 42 |
| | ** | |
| VEGI - 251 | EHELGLAFTKNRMNYTNKFLLIPESGDYFIYSQVTFRGMTSECSEIRQAGRPNKPDSITV | 179 |
| VEGI - 192A | EHELGLAFTKNRMNYTNKFLLIPESGDYFIYSQVTFRGMTSECSEIRQAGRPNKPDSITV | 120 |
| VEGI - 192B | EHELGLAFTKNRMNYTNKFLLIPESGDYFIYSQVTFRGMTSECSEIRQAGRPNKPDSITV | 120 |
| VEGI - 174 | EHELGLAFTKNRMNYTNKFLLIPESGDYFIYSQVTFRGMTSECSEIRQAGRPNKPDSITV | 102 |
| VEGI - 251 | VITKVTDSYPEPTQLLMGTKSVCEVGSNWFQPIYLGAMFSLQEGDKLMVNVSDISLVDYT | 239 |
| VEGI - 192A | VITKVTDSYPEPTQLLMGTKSVCEVGSNWFQPIYLGAMFSLQEGDKLMVNVSDISLVDYT | 180 |
| VEGI - 192B | VITKVTDSYPEPTQLLMGTKSVCEVGSNWFQPIYLGAMFSLQEGDKLMVNVSDISLVDYT | 180 |
| VEGI - 174 | VITKVTDSYPEPTQLLMGTKSVCEVGSNWFQPIYLGAMFSLQEGDKLMVNVSDISLVDYT | 162 |
| VEGI - 251 | KEDKTFFGAFLL | 251 |
| VEGI - 192A | KEDKTFFGAFLL | 192 |
| VEGI - 192B | KEDKTFFGAFLL | 192 |
| VEGI - 174 | KEDKTFFGAFLL | 174 |

* VEGI-174 (SEQ ID NO: 6) is referred to previously as VEGI (GenBank accession number AF039390)
** Homologous sequence in all isoforms begins at V₂₄ of VEGI-174 (SEQ ID NO:6), V₁₀₁ of VEGI-251(SEQ ID NO: 4, V₄₂ of VEGI-192A (SEQ ID NO:1), and V₄₂ of VEGI-192B (SEQ ID NO:5).

Variants of VEGI of the present invention may include one or more amino acid substitutions, deletions or additions that do not significantly change the activity of the protein. Variants may be from natural mutations or human manipulation. Changes can be of a minor nature, such as conservative amino acid substitutions that do not significantly affect the folding or activity of the protein. To improve or alter the characteristics of VEGI polypeptides, protein engineering may be employed. Recombinant DNA technology known to those skilled in the art can be used to create novel mutant proteins or mutants including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Thus, VEGI also encompasses VEGI derivatives and analogs that have one or more amino acid residues deleted, added, or substituted to generate VEGI polypeptides that are better suited for expression, scale up, etc., in the host cells chosen. In some embodiments, amino acid sequences of the VEGI variants are at least about any of 70%, 75%, 80%, 85%, 90%, 95%, 96%, 97%, 98%, or 99% identical to a VEGI (such as from a mammalian, a human VEGI).

Two polypeptide sequences are said to be “identical” if the sequence of amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison,. Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11: 105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by 100 to yield the percentage of sequence identity.

Variants of VEGI also encompass fusion proteins comprising VEGI polypeptides. Biologically active VEGI polypeptides can be fused with sequences, such as sequences that enhance immunological reactivity, facilitate the coupling of the polypeptide to a support or a carrier, or facilitate refolding and/or purification (e.g., sequences encoding epitopes such as Myc, HA derived from influenza virus hemagglutinin, His-6, FLAG, or the His-Tag shown in Table 3). These sequences may be fused to VEGI polypeptide at the N-terminal end or at the C-terminal end. In addition, the protein or polynucleotide can be fused to other or polypeptides which increase its function, or specify its localization in the cell, such as a secretion sequence. Methods for producing recombinant fusion proteins described above are known in the art. The recombinant fusion protein can be produced, refolded and isolated by methods well known in the art. In some embodiments, the VEGI protein used a fusion polypeptide comprising histidine residues, which may be prepared as described in the Examples. In some embodiments, the histidine fusion protein comprises SEQ ID NO:3.

Variants of VEGI also include functional equivalent variants. Functional equivalent variants are identified and characterized by any (one or more) of the following criteria: (a) ability to inhibit endothelial cell growth and/or proliferation; b) ability to induce endothelial cell death; b) ability to inhibit angiogenesis; c) ability to inhibit tumor growth (e.g., breast and lung cancer); d) ability to activate host immune system, for example ability to induce production of one or more cytokines (such as IL-15 and IP-10). Biological activity of variants of VEGI may be tested using methods known in the art and methods described herein. In some embodiments, functional equivalent variants have at least about any of 50%, 60%, 70%, 75%, 80%, 85%, 90%, or 95% of activity as compared to full length native VEGI with respect to one or more of the biological assays described above (or known in the art).

The methods of the invention are typically practiced utilizing inclusion bodies containing VEGI polypeptide, such as are formed in bacterial (e.g., E. coli) cells which have been engineered to produce VEGI (such as VEGI-192A), as the starting material, but any source of denatured VEGI protein may be used. The VEGI may be from any species desired, and from any natural or non-natural VEGI sequence, according to the practitioner's preference. The full coding sequence of human VEGI gene are published in PCT WO 99/23105, WO03/039491 and U.S. patent application Pub. No. 2003/0170242. Additionally, altered VEGI (such as VEGI-192A) genes, such as genes with “silent” changes which improve expression in the host organism (“optimized” sequences), or genes encoding mutant VEGI with one or more amino acid sequence changes may also be used.

Recombinant (e.g., bacterial, such as E. coli) host cells may be engineered to produce VEGI polypeptide using any convenient technology. Most commonly, a DNA sequence encoding the desired VEGI is inserted into the appropriate site in a plasmid-based expression vector which provides appropriate transcriptional and translational control sequences, although expression vectors based on bacteriophage genomic DNA are also useful. It is generally preferred that the transcriptional control sequences are inducible by a change in the environment surrounding the host cells (such as addition of a substrate or pseudosubstrate to which the transcriptional control sequences are responsive), although constitutive transcriptional control sequences are also useful. As is standard in the art, it is also preferred that the expression vector include a positive selectable marker (e.g., the β-lactamase gene, which confers resistance to ampicillin) to allow for selection against bacterial host cells which do not contain the expression vector.

The bacterial host cells are typically cultured in a liquid growth medium for production of VEGI polypeptide under conditions appropriate to the host cells and expression vector. Preferably, the host cells are cultured in a bacterial fermenter to maximize production, but any convenient method of culture is acceptable (e.g., shaken flask, especially for cultures of less than a liter in volume). As will be apparent to those of skill in the art, the exact growing conditions, timing and rate of media supplementation, and addition of inducing agent (where appropriate) will vary according to the identity of the host cells and the expression construct.

After the bacterial host cells are cultured to the desired density (and after any necessary induction of expression), the cells are collected. Collection is typically conveniently effected by centrifugation of the growth medium, although any other convenient technique may be used. The collected bacterial host cells may be washed at this stage to remove traces of the growth medium, most typically by resuspension in a simple buffer followed by centrifugation (or other convenient cell collection method). At this point, the collected bacterial host cells (the “cell paste”) may be immediately processed in accordance with the invention, or it may be frozen for processing at a later time.

The cells of the cell paste are lysed to release the VEGI polypeptide-containing inclusion bodies. Preferably, the cells are lysed under conditions in which the cellular debris is sufficiently disrupted that it fails to appear in the pellet under low speed centrifugation. Commonly, the cells are suspended in a buffer at about pH 5 to 9, preferably about 6 to 8, using an ionic strength of the order of about 0.01 M to 2 M preferably about 0.1-0.2 M (it is apparently undesirable to use essentially zero ionic strength). Any suitable salt, including NaCl can be used to maintain an appropriate ionic strength level. The cells, while suspended in the foregoing buffer, are then lysed by techniques commonly employed such as, for example, mechanical methods such as freeze/thaw cycling, the use of a Manton-Gaulin press, a French press, or a sonic oscillator, or by chemical or enzymatic methods such as treatment with lysozyme. It is generally desirable to perform cell lysis, and optionally bacterial cell collection, under conditions of reduced temperature (i.e., less than about 20° C.).

Inclusion bodies are collected from the lysed cell paste using any convenient technique (e.g., centrifugation), then washed. If desired, the collected inclusion bodies may be washed. Inclusion bodies are typically washed by resuspending the inclusion bodies in a wash buffer, typically the lysis buffer, preferably with a detergent added (e.g., 1% TRITON X-100®), then recollecting the inclusion bodies. The washed inclusion bodies are then dissolved in solubilization buffer. Solubilization buffer comprises a high concentration of a chaotroph, a pH buffer that buffers the solution to a high pH, and one or more reducing reagents. The solubilization buffer may optionally contain additional agents, such as redox reagents, cation chelating agents and scavengers to neutralize protein-damaging free-radicals.

The instant invention utilizes urea as an exemplary chaotroph in the solubilization buffer, although guanidine hydrochloride (guanidine HCl) may also be used. Useful concentrations of urea in the solubilization buffer include about 7.5 M to about 9 M, about 8 M to about 8.5M, or about 8 M. When the chaotroph is guanidine HCl, useful concentrations include about 5 M to about 7 M, or about 5.5 M to about 6.5 M, or about 6 M.

The pH of the solubilization buffer is high, viz., in excess of pH 8.0, for example pH 9.0. Useful pH levels in the solubilization buffer are in the range of about 8.0 to about 11.0, about 9.0 to about 11.0, about 9.5 to about 10.5, about 10.0 to about 10.5, about 10, about 10.5, or about 10.8. As will be apparent to those of skill in the art, any pH buffering agent (or combination of agents) which effectively buffer at high pH are useful, although pH buffers which can buffer in the range of about pH 8 to about pH 9 or 10 are particularly useful. Useful pH buffering agents include tris(tris(hydroxymethyl)aminomethane), bicine (N,N-Bis(2-hydroxyethyl)glycine), HEPBS (2-Hydroxy-1,1-bis[bydroxymethyl]ethyl)amino]-1-propanesulfonic acid), TAPS ([(2-Hydroxy-1,1 -bis[bydroxymethyl]ethyl)amino]-1-propanesulfonic acid), AMPD (2-Amino-2-methyl-1,3-propanediol).N-(2-Hydroxyethyl)piperazine-N′-(4-butanesulfonic acid)), and the like. The pH buffering agent is added to a concentration that provides effective pH buffering, such as from about 50 to about 150 mM, about 75 mM to about 125 mM, or about 100 mM.

Reducing reagents are included in the solubilization buffer to reduce disulfide bonds and maintain cysteine residues in their reduced form. Useful reducing reagents include β-mercaptoethanol, dithiothreitol, and the like. Additionally, the solubilization buffer may contain disulfide reshuffling or “redox” reagents (e.g., a combination of oxidized and reduced glutathione). When the redox reagents are oxidized and reduced glutathione (GSSG and GSH, respectively), the inventor has found that useful concentrations include about 0.1 mM to about 11 mM and useful ratios include about 10:1, about 5:1, and about 1:1 (GSH:GSSG).

The solubilization buffer may contain additional components. For example, the solubilization buffer may contain a cation chelator such as a divalent cation chelator like ethylenediaminetetraacetic acid (EDTA) or ethylene glycol-bis(2-aminoethylether)-N,N,N′,N′-tetraacetic acid (EGTA). EDTA or EGTA is added to the solubilization buffer at a concentration of about 0.5 to about 5 mM, and commonly at about 1 mM. Additionally, a free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM) and other amino acids and amines.

An exemplary solubilization buffer comprises about the following concentrations of the following components: 8M urea, 0.1 M Tris, 1 mM glycine, 10 mM beta-mercaptoethanol, 10 mM dithiothreitol (DTT), 1 mM reduced glutathion (GSH), 0.1 mM oxidized glutathion (GSSG), pH 10.5 or 10.8.

The inclusion body/solubilization buffer mixture is incubated to allow full solubilization. The incubation period is generally from about six hours to about 24 hours, and more commonly about eight to about 14 hours or about 12 hours. The inclusion body/solubilization buffer mixture incubation may be carried out at reduced temperature, commonly at about 4° to about 10° C.

After the incubation is complete, the inclusion body/solubilization buffer mixture is clarified to remove insoluble debris. Clarification of the mixture may be accomplished by any convenient means, such as filtration (e.g., by use of depth filtration media) or by centrifugation. Clarification should be carried out at reduced temperature, such as at about 4° to about 10° C.

The clarified mixture is then diluted using the same solubilization buffer to achieve the appropriate protein concentration for refolding. Protein concentration may be determined using any convenient technique, such as Bradford assay, light absorption at 280 nm (A₂₈₀), and the like. The inventor has found that a solution having an A₂₈₀ of about 2.0 (approximately 1.7 mg/mL) to about 10.0 (e.g., about any of 2.0, 3.0, 4.0, 5.0, 6.0, 7.0, 8.0, 9.0, and 10.0) is appropriate for use in the instant methods. If desired, this mixture may be held, refrigerated (e.g. at 4° C.), for later processing, although the mixture is not normally held for more than about four weeks.

The concentration-adjusted inclusion body solution is first rapidly diluted about 20 fold with refolding buffer. The dilution is performed by adding inclusion body solution into the refolding buffer. The inclusion body solution may be diluted about 10 to about 100 fold, about 10 to about 50 fold, about 10 to about 25 fold, about 15 to about 25 fold with refolding buffer. The inclusion body solution is diluted to reduce urea and protein concentration. The final protein concentration after dilution may be about 0.01 mg/ml to about 1 mg/ml, about 0.1 mg/ml to about 0.5 mg/ml. The refolding buffer contains one or more detergents and a pH buffer. The refolding buffer may also contain a low concentration of chaotroph, a disulfide reshuffling reagent, and a divalent cation chelator. The refolding buffer may include additional agents, such as free-radical scavengers. “Rapid” dilution, within the context of the invention means over a period of less than about 25 minutes, and the dilution process is generally carried out during periods of about two minutes to about 25 minutes, or about five to about 20 minutes. The diluted solubilized VEGI (such as VEGI-192A) solution is typically held for one to two hours following the completion of the rapid dilution process.

One or more detergents are included in the refolding buffer. Examples of detergents that can be used are sodium lauroyl sarcosine, trimethylamine N-oxidedihydrate (TMAO), cetyltrimethylammunium bromide (CTAB). For example, sodium lauroyl sarcosine may be used at about 0.004% to about 0.1%. Ionic detergents may be used, such as cholic acid and its derivatives, sodium N-dodecyl sulfate (SDS), and TOPPS. Other detergents, such as zwitterionic detergents, may also be used.

The pH of the refolding buffer may be the same as the solubilization buffer. The pH buffering agent in the refolding buffer may be any buffering agent or combination of buffering agents that are effective pH buffers at pH levels of about 8 to about 9 or about 10 or about 10.5. Useful pH buffering agents include tris(tris(hydroxymethyl)aminomethane), bicine (N,N-Bis(2-hydroxyethyl)glycine), HEPBS (2-Hydroxy-1,1-bis[bydroxymethyl]ethyl)amino]-1-propanesulfonic acid), TAPS ([(2-Hydroxy-1,1-bis[bydroxymethyl]ethyl)amino]-1-propanesulfonic acid), and AMPD (2-Amino-2-methyl-1,3-propanediol).N-(2-Hydroxyethyl)piperazine-N′-(4-butanesulfonic acid)). The pH buffering agent is added to a concentration that provides effective pH buffering, such as from about 10 to about 150 mM, about 50 to about 150 mM, about 75 mM to about 125 mM, or about 100 mM.

The redox reagents included in the refolding buffer must be effective in ‘shuffling’ cysteine sulfhydryl groups between their oxidized and reduced states. The redox environment of the refolding reaction may be adjusted by manipulating the concentration of the redox reagents. When the redox reagents are oxidized and reduced glutathione (GSSG and GSH, respectively), the inventor has found that useful concentrations include about 0.005 mM to about 0.05 mM, about 0.1 mM to about 11 mM and useful ratios include about 10:1, about 5:1, and about 1:1 (GSH:GSSG).

The divalent cation chelator may be any molecule that effectively chelates Ca⁺⁺ and other divalent cations. Exemplary cation chelators for use in the refolding buffer include ethylenediaminetetraacetic acid (EDTA) or ethylene glycol-bis(2-aminoethylether)-N,N,N′,N′-tetraacetic acid (EGTA). When EDTA or EDTA is the divalent cation chelator, it is added to the refolding buffer at a concentration of about 0.5 to about 5 mM, and commonly at about 1 mM.

Additional components useful in the refolding buffer include free-radical scavengers. A free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM).

An exemplary refolding buffer comprises Tris, Sodium Lauroyl Sarcosine, Trimethylamine N-oxidedihydrate, and Cetyltrimethylammonium Bromide. For example, the refolding buffer may comprise about 0.034 mM to about 1.36 mM Sodium Lauroyl Sarcosine, about 0.009 mM Trimethylamine N-oxidedihydrate, and about 0.005 mM Cetyltrimethylammonium Bromide.

The pH of the refolding solution is then slowly reduced from elevated pH to near neutral pH using an appropriate acid. The time period for pH reduction can range from about 20-24 hours to about 10 days, about 20 to about 50 hours, about 20 to about 40 hours, about 20 to about 30 hours, about 24 to about 40 hours. The time period for pH reduction can be at least about 20 hours, about 24 hours, about 30 hours, about 40 hours, about 48 hours, about 50 hours, about 4 days, about 5 days. In some embodiments, the time period for pH reduction is about 2-5 days. Appropriate acids for pH adjustment will depend on the pH buffer used in the refolding buffer. For example, when the pH buffering agent is tris, the pH should be adjusted with hydrochloric acid (HCl).

Following completion of pH adjustment, the refolding reaction is incubated for a period of about one to two hours to about 18 to 24 hours. The refolding reaction may be carried out at room temperature (e.g., about 18-20° C.) or at slightly reduced temperatures (e.g., about 14-16° C.), depending on the preferences of the practitioner and the available facilities.

Following the refolding reaction, properly refolded VEGI (such as VEGI-192A) may be concentrated and further purified. Concentration of the refolded protein may be accomplished using any convenient technique, such as ultrafiltration, diafilitration, chromatography (e.g., ion-exchange, hydrophobic interaction, or affinity chromatography) and the like. Where practical, it is preferred that concentration be carried out at reduced temperature (e.g., about 4-10° C.). The concentration step may also include a buffer exchange process to remove the detergent in the refolding buffer before purifying the protein.

While any convenient protein purification protocol may be used. In some embodiments, two types of chromatography may be used for purification. For example, size exclusion chromatography (SEC) and affinity chromatography may be used.

Size exclusion chromatography (SEC) may be performed using any convenient chromatography medium which separates properly folded VEGI (such as VEGI-192A) from unfolded VEGI and multimeric VEGI. The inventor has found that media having the ability to size fractionate proteins of about 104 to about 6×10⁵ daltons (globular proteins) are useful for this step. Exemplary SEC media include Sephacryl® 300 and Superdex™ 200. This step may also be used to perform buffer exchange, if so desired. The exact conditions for SEC will depend on the exact chromatography media selected, whether buffer exchange is to be accomplished, the requirements of any later purification steps, and other factors known to those of skill in the art.

The properly folded VEGI (such as VEGI-192A) may be further purified utilizing affinity chromatography, for example, Ni⁺-chelating column for VEGI-192A with His-Tag shown in Examples.

Biological activity of VEGI produced from the properly folded recombinant VEGI produced in accordance with the invention may be measured using any acceptable assay method known in the art. See PCT WO03/039491; U.S. patent application Pub. No. 2003/0170242; U.S. patent application Pub. No. 2002/0111325. An exemplary method of measuring VEGI activity is described herein in Example 2, which measures inhibition of vascular endothelial cell proliferation in vitro. Other assays include administering VEGI into animals bearing tumor and measuring tumor growth in the animals. Tumor bearing animals (e.g., lung and breast tumor) are known in the art. For example, human breast cancer xenograft tumor growth model generated by injecting MDA-MB-231 human breast cancer cells into the mammary fat pads of a female athymic nude mouse and murine Lewis lung carcinoma primary tumor model generated by implanting by subcutaneous injection of murine Lewis lung cancer cells (ATCC) on the back of a black mouse (C57BL) may be used to test the activity of VEGI. See U.S. patent application Pub. No. 2003/0170242; O'Reilly et al., Cell 79(2):315-28 (1994); Cao et al., J. Clin. Invest. 101(5): 1055-63 (1998). In some embodiments, VEGI polypeptides produced have an IC₅₀ of at least about 1000 ng/ml, about 100 ng/ml, about 60 ng/ml, about 40 ng/ml, about 20 ng/ml, about 12 ng/ml, or about 6 ng/ml in inhibiting vascular endothelial cell proliferation in vitro.

As is well understood in the art, all concentrations and pH values need not be exact and reference to a given value reflects standard usage in the art, does not mean that the value cannot vary.

The following examples provide a detailed description of the production of properly folded recombinant VEGI in accordance with the methods of the invention and the characterization thereof. These examples are not intended to limit the invention in any way.

### EXAMPLES

### Example 1

### Refolding and Purification of Recombinant VEGI-192A and other VEGI

Vector construction and expression: A DNA fragment encoding human VEGI-192A as shown in Table 1 was produced by PCR amplification. The PCR product was inserted into the Nde I/Bam H1 sites of pET19b (Cat. No. 69677-3, Novagen, San Diego, Calif.), producing a VEGI-192A protein with an N-terminal fusion tag (Table 3). The PCR product was also inserted into the NdeI/BamHI sites of pET-11 (Novagen), producing a VEGI-192A protein without an N-terminal fusion tag. After PCR, ligation, and transformation into the BL21 (DE3) strain of E. coli, single colonies were selected and amplified and then ultimately the construct was sequenced to assure the correctness of the DNA sequence. The nucleotide sequence and amino acid sequence of VEGI-192A with a N-terminal His-Tag are shown in Table 3. The amino acid sequence of VEGI-192A without His-Tag is shown in Table 1.

TABLE 3

Nucleotide sequence and amino acid sequence of human
VEGI-192A with a N-terminal His-Tag

Gene coding sequence of VEGI-192A with a N-terminal fusion tag (SEQ ID NO:2):
ATGGGCCATCATCATCATCATCATCATCATCATCACAGCAGCGGCCATATCGACGACGA
CGACAAGCATATGCAACTCACAAAGGGCCGTCTTCATTTCAGTCACCCTTTGTCTCATA
CAAAGCACATTTCTCCTTTTGTTACAGATGCACCTCTTAGAGCAGACGGAGATAAGCCA
AGGGCACACCTGACAGTTGTGAGACAAACTCCCACACAGCACTTTAAAAATCAGTTCCC
AGCTCTGCACTGGGAACATGAACTAGGCCTGGCCTTCACCAAGAACCGAATGAACTAT
ACCAACAAATTCCTGCTGATCCCAGAGTCGGGAGACTACTTCATTTACTCCCAGGTCAC
ATTCCGTGGGATGACCTCTGAGTGCAGTGAAATCAGACAAGCAGGCCGACCAAACAAG
CCAGACTCCATCACTGTGGTCATCACCAAGGTAACAGACAGCTACCCTGAGCCAACCCA
GCTCCTCATGGGGACCAAGTCTGTATGCGAAGTAGGTAGCAACTGGTTCCAGCCCATCT
ACCTCGGAGCCATGTTCTCCTTGCAAGAAGGGGACAAGCTAATGGTGAACGTCAGTGAC
ATCTCTTTGGTGGATTACACAAAAGAAGATAAAACCTTCTTTGGAGCCTTCTTACTATAG
Protein sequence of VEGI-192A with a N-terminal His-Tag (SEQ ID NO:3):
MGHHHHHHHHHHSSGHIDDDDKHMQLTKGRLHFSHPLSHTKHISPFVTDAPLRADGDKPR
AHLTVVRQTPTQHFKNQFPALHWEHELGLAFTKNRMNYTNKFLLIPESGDYFIYSQVTFRG
MTSECSEIRQAGRPNKPDSITVVITKVTDSYPEPTQLLMGTKSVCEVGSNWFQPIYLGAMFS
LQEGDKLMVNVSDISLVDYTKEDKTFFGAFLL

To produce VEGI-192A protein (having His-Tag or no His-Tag), VEGI-192A expression vector was transfected into BL21 (DE3) strain of E. coli and plated on ZB plates with ampicillin. A single colony was selected and used to inoculate 100 mL of ZB media (10 g/l NZ amine A (Sigma) and 5 g/l NaCl) with ampicillin and grown overnight (approximately 16 hours) at 30° C. The 20 mL of the 100 mL starter culture was then used to inoculate 1 L of LB media with ampicillin, and the culture was incubated at 37° C. with shaking until the optical density at 600 nm (OD₆₀₀) reached 0.4-0.6. Isopropyl-beta-D-thiogalactopyranoside (IPTG) was then added to 0.5 mM to induce VEGI-192A expression, and the culture was incubated a further three hours with shaking. Large scale expression was accomplished utilizing multiple IL shaker flasks at 37° C.

Processing of Inclusion Bodies Before Refolding: The inclusion bodies were harvested from bacterial. Bacterial cells were collected by centrifugation, then resuspended in 20 mL of TN per 1 L of bacteria culture (150 mM NaCl, 50 mM tris, pH 8.0) with 1% TRITON X-100®. Ten milligrams of lysosyme was added, and the cell suspension was frozen at −20° C. overnight. The lysate was then thawed and 20 μL of 1 M magnesium sulfate and 100 μg of DNase were added. The cells were incubated, with stirring, until the released bacterial DNA was completely dissolved. The lysate was then diluted with 250 mL of TN with 1% TRITON X-100® and the mixture was stirred for 2-4 hours. Inclusion bodies were collected by centrifugation, and washed three times (by resuspension and centrifugation).

Refolding and Chromatographic Isolation of Refolded Protein (VEGI-192A with His-Tag): The washed inclusion bodies were dissolved in 8M urea, 0.1 M Tris, 1 mM glycine, 10 mM beta-mercaptoethanol, 10 mM dithiothreitol (DTT), 1 mM reduced glutathion (GSH), 0.1 mM oxidized glutathion (GSSG), pH 10.5. In another experiment, pH 10.8 was used. The absorbance at 280 nm (OD280) of the protein solution was adjusted to 2.0. The solution was clarified by ultracentrifugation (30 minutes×66,000 g), then refolded.

The clarified solution was rapidly diluted into 20 volumes of 20 mM Tris, 1.36 mM Sodium Lauroyl Sarcosine, 0.009 mM Trimethylamine N-oxidedihydrate, 0.005 mM Cetyltrimethylammonium Bromide, pH 10.5. The pH of 10.8 was also used for the clarified solution in another experiment. The resulting solution was adjusted to pH 8.0 with 1 M HCl stepwise over a 4-day period. The pH adjustment was also, performed over a 2-5 day period in another experiment.

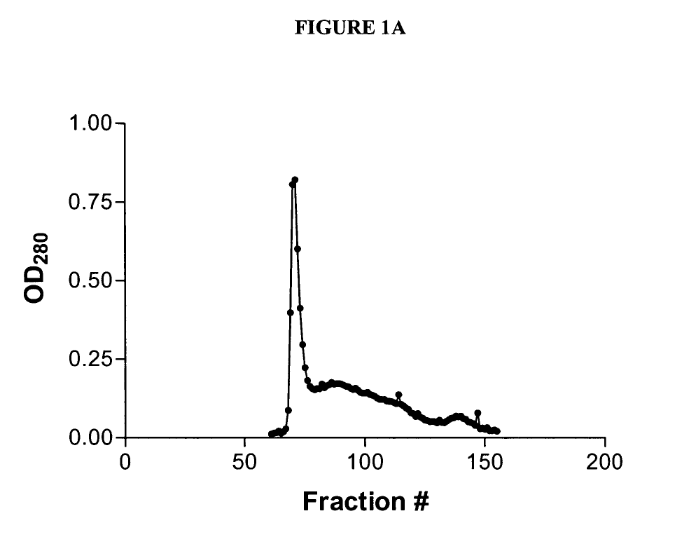

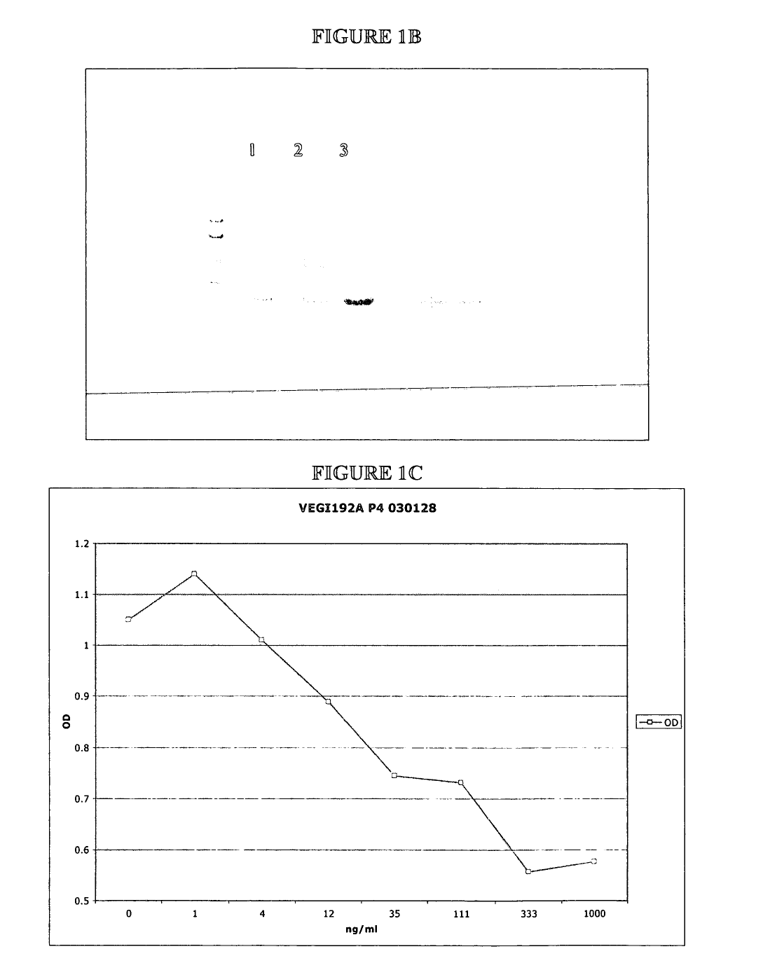

The refolded proteins were concentrated by ultrafiltration (Millipore Pellicon, 10,000 Da cut-off membrane and applied to a Sephacryl S-300 column equilibrated and eluted with 20 mM Tris, 0.4 M urea, 0.2 M NaCl, pH 8 (FIG. 1). Fractions from FIG. 1A were pooled (shown as 1, 2, 3) and concentrated to about 5 mg/ml (SDS-PAGE shown in FIG. 1B) for endothelial cell arrest assay described in Example 2.

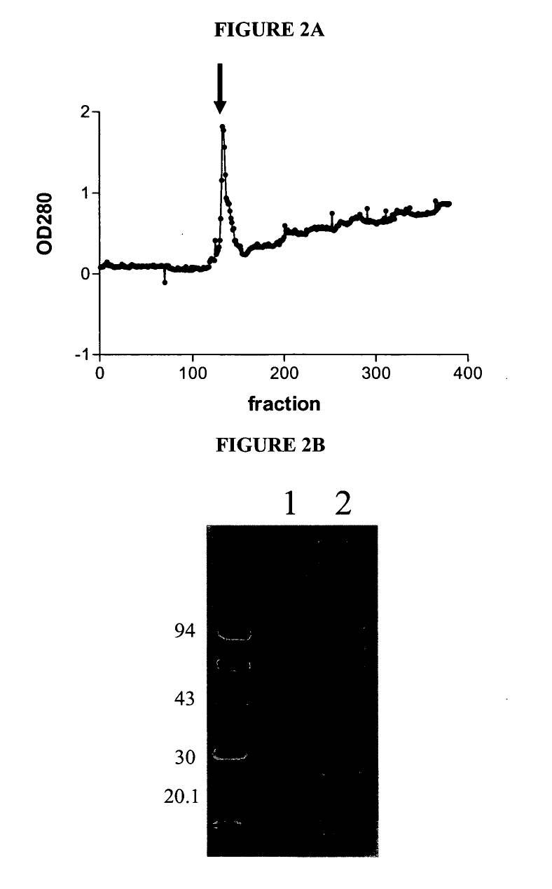

Functional VEGI-192A was also purified using Ni⁺-Affinity column. Ni⁺-chelating column (500 ml) was equilibrated with 20 mM Tris, 1.36 mM Sodium Lauroyl Sarcosine, 0.4 M urea, pH 8. Refolded VEGI-192A (4 L) was applied to the column, and the column was washed with 1 L of 20 mM Tris, 0.4 M urea, 0.2 M NaCl, 5 mM Immidozole, pH 8 (buffer A). VEGI-192A was eluted from the column with a linear gradient of immidozole (5 to 500 mM) in buffer A. The eluted peak shown in FIG. 2A was pooled, and dialyzed against 20 mM Tris, 0.4 M urea, 0.2 M NaCl, pH 8. The dialyzed VEGI-192A was then concentrated by ultrafiltration to about 5 mg/ml. The non-reduced and reduced SDS-PAGE analysis of produced VEGI-192A is shown in FIG. 2B.

In another experiment, the refolding process was performed by rapidly diluting the clarified solution containing the solubilized VEGI-192A into 20 volumes of 20 mM Tris, 0.034 mM Sodium Lauroyl Sarcosine, 0.009 mM Trimethylamine N-oxidedihydrate, 0.005 mM Cetyltrimethylammonium Bromide, pH 10.5. The pH of 10.8 was also used. The resulting solution was adjusted to pH 8.0 with 1 M HCl stepwise over a 4-day period. The refolded VEGI-192A was concentrated and purified the same way as described above using S-300 column chromatography. The activity of the purified VEGI-192A using this refolding buffer was 100 times less than the refolding condition described above, determined by the endothelial cell arrest assay described in Example 2.

Refolding and Chromatographic Isolation of Refolded Protein (VEGI-192A without His-Tag): The washed inclusion bodies were dissolved in 8M urea, 0.1 M Tris, 1 mM glycine, 10 mM beta-mercaptoethanol, 10 mM dithiothreitol (DTT), 1 mM reduced glutathion (GSH), 0.1 mM oxidized glutathion (GSSG), pH 10.8. The absorbance at 280 nm (OD280) of the protein solution was adjusted to 2.0. The solution was clarified by ultracentrifugation (30 minutes×66,000 g), then refolded.

The clarified solution was rapidly diluted into 20 volumes of 20 mM Tris, 1.36 mM Sodium Lauroyl Sarcosine, 0.009 mM Trimethylamine N-oxidedihydrate, 0.005 mM Cetyltrimethylammonium Bromide, pH 10.8. The resulting solution was adjusted to pH 8.0 with 1 M HCl stepwise over a 4-day period.

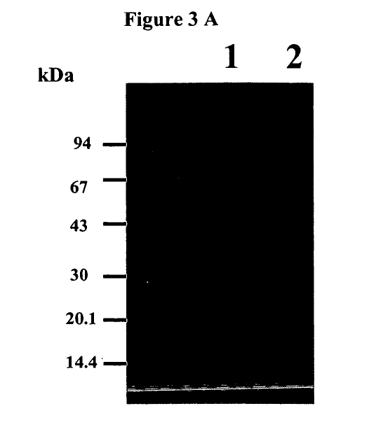

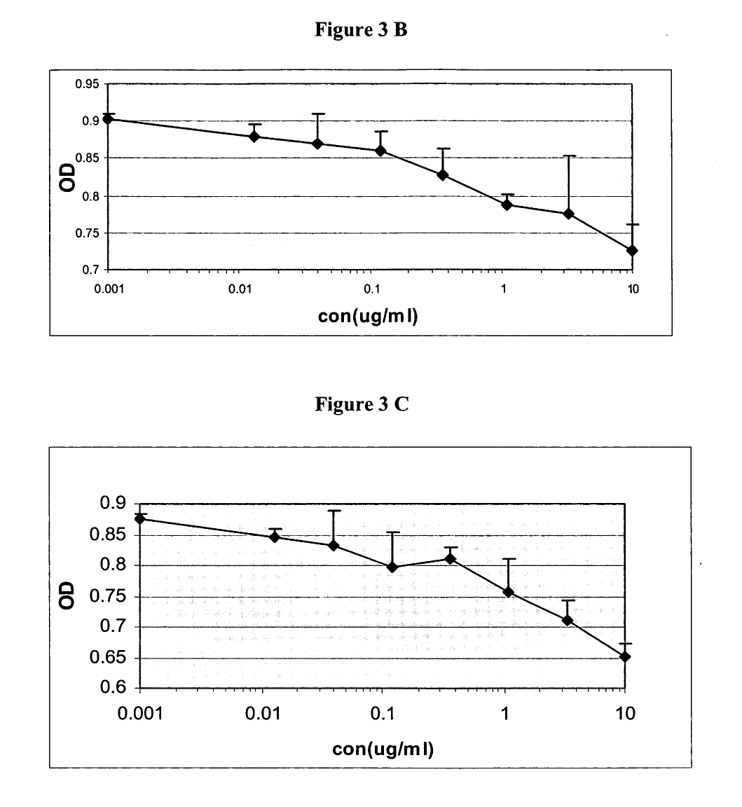

The refolded proteins were concentrated by ultrafiltration (Millipore Pellicon, 10,000 Da cut-off membrane and applied to a Sephacryl S-300 column equilibrated and eluted with 20 mM Tris, 0.4 M urea, 0.2 M NaCl, pH 8. Fractions were pooled and concentrated to about 5 mg/ml (SDS-PAGE shown in FIG. 3A, lane 1) for endothelial cell arrest assay described in Example 2.

Refolding of VEGI-174 and VEGI-251 fragment (amino acids 86-251): A DNA fragment encoding human VEGI-174 was produced by PCR amplification and the PCR product was inserted into the NdeI/BamH1 sites of pET-19b, producing a VEGI-174 with an N-terminal His-Tag. A DNA fragment encoding amino acids 86-251 of VEGI-251 was also produced by PCR amplification and the PCR product were inserted into the NdeI/Bam H1 sites of pET11, producing a VEGI-251 fragment (amino acids 86-251) (without any tag). After producing VEGI-174 and VEGI-251 fragment in E. coli, these proteins were refolded and purified as described above for VEGI-192A (with His-Tag). In endothelial cell growth assay, these proteins demonstrated activity in inhibiting endothelial cell proliferation in vitro with IC₅₀ in the range of 2.4 to 24 nM.

### Example 2

### Characterization of Biological Activity of Recombinant VEGI-192A by Endothelial Cell Growth Arrest Assay

Adult bovine aortic endothelial (ABAE) cells were cultured in IMEM (Gibco Biofluids, Rockville, Md.), 10% FBS, 1 ng/ml fibroblast growth factor-2 (Promega, Madison, Wis.), 37° C., 5% CO₂. The extent of quiescence of the cells was determined by ³H-thymidine incorporation. Cells were considered synchronized at G₀ phase of the cell cycle if no more than 5% of the cells were incorporating ³H-thymidine. The G₀-synchronized cells re-entered the growth cycle when they were re-seeded scarcely (5000 cells/cm2) in IMEM with 10% FBS and 1 ng/ml fibroblast growth factor-2, and incubated at 37° C., 5% CO₂. VEGI-192A preparations were added to the culture media either at the time of seeding the cells or at a time point when the cells entered the growth cycle in 20 hours post seeding. Single cell suspension was prepared from each culture well at a given time interval by trypsinization. The number of cells in each suspension was determined by using a Coulter counter, or by using a colorimetric assay utilizing a tetrazolium compound MTS (Promega) that can be metabolized by living cells to generate a blue colored compound detectable at 490 nm; the metabolic rate of MTS was proportional to the number of living cells in culture. Concentrations of VEGI-192A utilized in this assay ranged from 0.1 ng/mL to 1 μg/mL or from 1 ng/mL to 10 μg/mL.

In ABAE endothelial cell arrest assay using a colorimetric assay with MTS, all three pools of VEGI-192A (with His-Tag) from FIG. 1A were active with pool 3 being the most active (FIG. 1C). As shown in FIG. 1C, recombinant VEGI-192A (with His-Tag) in pool 3 exhibited an IC₅₀ of 12 ng/ml (0.49 nM) in inhibiting endothelial cell proliferation in vitro. In another experiment, recombinant VEGI-192A (with His-Tag) produced exhibited an IC₅₀ of 0.24 nM (6 ng/ml) in inhibiting endothelial cell proliferation in vitro.

In ABAE endothelial cell arrest assay using a colorimetric assay with MTS, VEGI-192A (with no tag) was also active (FIG. 3B). Both refolded recombinant VEGI-192A (with no tag) (FIG. 3B) and VEGI-192A with N-terminal His-Tag (FIG. 3C) inhibited endothelial cell proliferation in vitro.

### Example 3

### Production of VEGI-192A in E. coli Using a Synthetic Gene

A synthetic nucleotide sequence (shown in Table 4) encoding VEGI-192A with an N-terminal His-Tag was used for expressing the protein in E. coli. The expression level (purified inclusion body yield) was significantly higher than the level expressed using the native gene. VEGI-192A (without any tag) was also produced using the synthetic gene (starting from the arrow position in Table 4) with significant higher yield.

TABLE 4
Nucleotide sequence of a synthetic gene (SEQ ID NO:7) encoding VEGI-192A.

catatgggccatcatcaccaccatcaccatcaccatcatagcagcggccatatcgatgat
zHzzMzzGzzH↓ HzzHzzHzzHzzHzzHzzHzzHzzHzzSzzSzzGzzHzzIzzDzzD
Gatgataaacacatgcagctgaccaaaggccgtctgcattttagccatccgctgagccat
zDzzDzzKzzHzzMzzQzzLzzTzzKzzGzzRzzLzzHzzFzzSzzHzzPzzLzzSzzH
accaaacatatcagcccgtttgttaccgatgcgccgctgcgtgcggatggtgataaaccg
zTzzKzzHzzIzzSzzPzzFzzVzzTzzDzzAzzPzzLzzRzzAzzDzzGzzDzzKzzP
cgtgcgcatctgaccgttgttcgtcagaccccgacccagcattttaaaaaccagtttccg
zRzzAzzHzzLzzTzzVzzVzzRzzQzzTzzPzzTzzQzzHzzFzzKzzNzzQzzFzzP
gcgctgcattgggaacatgaactgggtctggcgtttaccaaaaaccgcatgaactatacc
zAzzLzzHzzWzzEzzHzzEzzLzzGzzLzzAzzFzzTzzKzzNzzRzzMzzNzzYzzT
aacaaattcctgctgattccggaaagcggcgattatttcatctatagccaggtgaccttt
zNzzKzzFzzLzzLzzIzzPzzEzzSzzGzzDzzYzzFzzIzzYzzSzzQzzVzzTzzF
cgtggtatgaccagcgaatgcagcgaaattcgtcaggcgggtcgtccgaataaaccggat
zRzzGzzMzzTzzSzzEzzCzzSzzEzzIzzRzzQzzAzzGzzRzzPzzNzzKzzPzzD
agcatcaccgttgttatcaccaaagtgaccgatagctatccggaaccgacccagctgctg
zSzzIzzTzzVzzVzzIzzTzzKzzVzzTzzDzzSzzYzzPzzEzzPzzTzzQzzLzzL
atgggcaccaaaagcgtgtgtgaagttggcagcaattggtttcagccgatttatctgggc
zMzzGzzTzzKzzSzzVzzCzzEzzVzzGzzSzzNzzWzzFzzQzzPzzIzzYzzLzzG
gcgatgtttagcctgcaggaaggcgataaactgatggttaacgtgagcgatattagcctg
zAzzMzzFzzSzzLzzQzzEzzGzzDzzKzzLzzMzzVzzNzzVzzSzzDzzIzzSzzL
gtggattataccaaagaagataaaaccttcttcggcgcgttcctgctgtaaggatcc
zVzzDzzYzzTzzKzzEzzDzzKzzTzzFzzFzzGzzAzzFzzLzzLzz-zzGzzS

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

1. A method for producing a refolded recombinant VEGI polypeptide, comprising:
(a) solubilizing a denatured VEGI polypeptide with a solubilization buffer, said solubilization buffer comprising a high concentration of chaotroph, a reducing agent, and having a pH of about 9.0 to about 11.0, thereby produce a solubilized VEGI polypeptide solution,
(b) rapidly diluting said solubilized VEGI polypeptide solution with refolding buffer by adding the solubilized VEGI polypeptide solution into the refolding buffer comprising a detergent, thereby producing a diluted solubilized VEGI polypeptide solution, and
(c) reducing the pH of the diluted solubilized VEGI polypeptide solution to a pH of about 7.5 to about 8.5, wherein said pH reducing is carried out over a period of at least about 20 hours, thereby producing a refolded VEGI polypeptide.

2. The method of claim 1, wherein said VEGI polypeptide is a human VEGI.

3. The method of claim 2, wherein said VEGI polypeptide comprises amino acids 24-174 of SEQ ID NO:6.

4. The method of claim 2, wherein said VEGI polypeptide comprises amino acid sequence of SEQ ID NO:6.

5. The method of claim 2, wherein said VEGI polypeptide comprises amino acids 86-251 of SEQ ID NO:4.

6. The method of claim 1, wherein said chaotroph is urea.

7. The method of claim 6, wherein said urea is at about 8 M concentration.

8. The method of claim 1, wherein said chaotroph is guanidine hydrochloride.

9. The method of claim 8, wherein said guanidine hydrochloride is at about 6 M concentration.

10. The method of claim 1, wherein said solubilizing buffer is about pH 10.0 to about pH 10.8.

11. The method of claim 1, wherein the pH of the diluted solubilized VEGI polypeptide solution is reduced to about pH 8.0.

12. The method of claim 1, wherein the solubilization buffer comprises about 8 M urea, about 0.1 M Tris, about 1 mM glycine, about 10 mM β-mercaptoethanol, about 10 mM dithiothreitol (DTT), about 1 mM reduced glutathion (GSH), and about 0.1 mM oxidized glutathion (GSSG) at pH about 10.0 to about 10.8.

13. The method of claim 1, said method further comprising adjusting the A₂₈₀ of the solubilized VEGI polypeptide solution to about 2.0 to about 5.0 before step (b).

14. The method of claim 13, wherein the A₂₈₀ of the solubilized VEGI polypeptide solution is adjusted by diluting the solubilized VEGI polypeptide solution with the solubilization buffer.

15. The method of claim 1, wherein the solubilized VEGI polypeptide solution is diluted into about twenty-fold refolding buffer.

16. The method of claim 1, wherein the refolding buffer comprises a mixture of detergents.

17. The method of claim 1, wherein the detergent is sodium lauroyl sarcosine, trimethylamine N-oxidedihydrate (TMAO), cetyltrimethylammunium bromide (CTAB), or any combination of these detergents.

18. The method of claim 17, wherein the detergent is a combination of sodium lauroyl sarcosine, trimethylamine N-oxidedihydrate (TMAO), cetyltrimethylammunium bromide (CTAB).

19. The method of claim 1, wherein said solubilization buffer comprises about 8M urea, about 0.1 M Tris, about 1 mM glycine, about 10 mM beta-mercaptoethanol, about 10 mM dithiothreitol (DTT), about 1 mM reduced glutathion (GSH), about 0.1 mM oxidized glutathion (GSSG), about pH 10.5; wherein said refolding buffer comprises about 20 mM Tris, about 1.36 mM Sodium Lauroyl Sarcosine, about 0.009 mM Trimethylamine N-oxidedihydrate, about 0.005 mM Cetyltrimethylammonium Bromide, about pH 10.5.

20. The method of claim 1, further comprising lysing a bacterial host cell comprising the denatured VEGI polypeptide and collecting said denatured VEGI polypeptide before step (a).

21. The method of claim 20, further comprising washing said denatured VEGI polypeptide.

22. The method of claim 1, further comprising purifying said refolded VEGI polypeptide.

23. The method of claim 22, wherein said refolded VEGI polypeptide is purified by size exclusion chromatography.

24. The method of claim 22, wherein buffer exchange is performed to remove the detergent in the refolding buffer before purification.


SEQUENCE LISTING (6 sequences)

SEQ ID NO: 1 - protein, 192 aa, Homo sapiens
MQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2 - DNA, 648 bp, Artificial Sequence
  Synthesized VEGI-192A with a N-terminal fusion tag
atgggccatcatcatcatcatcatcatcatcatcacagcagcggccatatcgacgacgacgacaagcatatgcaactcacaaagggccgtcttcatttcagtcaccctttgtctcatacaaagcacatttctccttttgttacagatgcacctcttagagcagacggagataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactatag

SEQ ID NO: 3 - protein, 215 aa, Artificial Sequence
  Synthesized VEGI-192A with a N-terminal His-Tag
MGHHHHHHHHHHSSGHIDDDDKHMQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 4 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 5 - protein, 192 aa, Homo sapiens
METSQEHQGPSDIHRIPWSWGQRNSHAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 6 - protein, 174 aa, Homo sapiens
MRRFLSKVYSFPMRKLILFLVFPVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL